(19) United States
(12) Patent Application Publication
(10) Pub. No.: US 2009/0239250 A1
(43) Pub. Date: Sep. 24, 2009

(54) METHOD FOR THE ANALYSIS OF CELLS

(75) Inventors: David R. Klug, London (GB); de Mello Andrew, Middlesex (GB); Richard H. Templer, Surrey (GB); Paul Michael William French, West Sussex (GB); Mark Andrew Aquilla Neil, Oxford (GB); Oscar Ces, London (GB); Peter Joseph Jacques Parker, Dorking (GB); Keith Robert Willison, London (GB)

Correspondence Address: WARNER NORCROSS & JUDD LLP, 900 FIFTH THIRD CENTER, 111 LYON STREET, N.W., GRAND RAPIDS, MI, 49503-2487, US

(73) Assignee: Imperial Innovations Limited, London (GB)

(21) Appl. No.: 11/720,740

(22) PCT Filed: Dec. 1, 2005
(86) PCT No.: PCT/GB05/04602

§ 371 Date: Oct. 6, 2008

(30) Foreign Application Priority Data
Dec. 3, 2004 (GB) 0426609.4

(51) Int. Cl. C12Q 1/02; C12M 1/34

(52) U.S. Cl. 435/29; 435/288.7

(57) ABSTRACT

The present invention provides an improved method of analysing and obtaining reliable data about the plasma membrane of single cells, using a microfluidic cell analyser. The microfluidic cell analyser of the invention comprises a single cell trap, a manipulator arranged to manipulate the outer surface of a cell in the trap, a detection zone in communication with the single cell trap and a detector.

The present invention relates to a microfluidic cell analyser.

The analysis of populations of cells has provided important information and has led to a number of important medical, clinical and scientific developments. However, cells within a given population are heterogeneous. This leads to problems with the use of population averaged cell preparation techniques and has prompted the need for alternative ways of analysing cells. In particular, this has lead to the development of single cell analytic methods.

Single cell analytic methods are believed to overcome disadvantages associated with population averaged analytic methods. In particular, such methods allow more accurate information to be obtained, allowing a better understanding of cell responses to therapeutic interventions and disease states. Single cell analysis therefore has the potential to become an important tool for aiding and enabling the development of predictive and preventative medicine tailored to individual patients.

Microfluidic systems have previously been used to perform single cell analysis. However, such analysis has concentrated on the intracellular components of the cell. The analytic methods of the art have therefore required the location of a single cell into a separation channel, lysis of the cell, separation of the cellular components and analysis of the intracellular component of interest by capillary electrophoresis.

While the analysis of intracellular components of a cell may provide interesting biological information, the interaction of a cell with its surrounding environment is primarily determined by the plasma membrane.

The plasma membrane defines the extent of a cell and moderates the interaction of the cell with its external environment. It performs many roles including acting as a filter, allowing active transport, controlling the entry and exit of substances into and out of the cell, generating differences in ion concentrations between the interior and exterior of the cell and sensing external signals.

The plasma membrane is the subject of many disease states and a target for many therapeutic interventions. The plasma membrane is involved in the transduction of a large number of signaling pathways, is the entry and exit route for all cellular components and is the means by which cells interact with and communicate with their environment. The plasma membrane is therefore an important target for single cell analysis.

The plasma membrane is a lipid bilayer, said bilayer being composed of phospholipids, cholesterol and glycolipids. In addition, the plasma membrane comprises a numerous additional components such as proteins, glycoproteins etc. The plasma membrane therefore contains many thousands of proteins as well as a complex mix of lipids. Current analysis methods of the plasma membrane are broadly based on one of two strategies. The conventional protein analysis method is to take an ensemble of cells from tissue or culture, homogonise them, and then separate the components. An example of this is the MALDI-TOF proteomic approach, which uses two-dimensional gels, or liquid phase separation to isolate individual proteins and mass spectrometry to identify them. The 2D gel MALDI-TOF approach is however highly unsatisfactory for membrane proteins, and has limited utility due to problems with low fidelity, insensitivity and isolation difficulties. An alternative approach for the analysis of lipid composition of plasma membranes is electrospray ionisation mass spectrometry (ESI-MS), which allows the analysis of phospholipid structures. Similar techniques can be applied to membrane proteins however the proteins must initially be separated using a combination of isoelectric focusing (IEF) and SDS electrophoresis which often proves difficult and time consuming.

An alternative analysis method is the use of in vivo fluorescence microscopy. This method can be applied to single cells. However, fluorescence microscopy depends on specificity of antibody or transfection labeling. This is a major disadvantage of fluorescence microscopy and has limited the application of this technique to the examination of only two, or three proteins at one time.

There is therefore a need in the art for an improved method of analysing and obtaining reliable data about the plasma membrane of cells, in particular the plasma membrane of single cells.

The first aspect of the invention provides a microfluidic cell analyser comprising a single cell trap, a manipulator arranged to manipulate the outer surface of a cell in the trap, a detection zone in communication with the single cell trap and a detector.

The invention may be put into practice in various ways and a number of specific embodiments will be described by way of example to illustrate the invention with reference to the accompanying drawing:

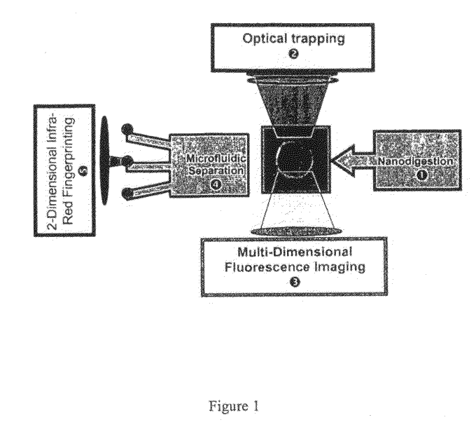

FIG. 1 which shows a schematic representation of the microfluidic cell analyser.

The microfluidic cell analyser as illustrated in FIG. 1 comprises a single cell trap, for example an optical trap (2) monitored by a multi-dimensional fluorescence imager (3), a manipulator (not shown) such as laser dissection, a microfluidic separator (4), a detection zone (not shown) and a detector (5).

Pre-separation of the cell sample is carried out at (1) by controlled microfluidic nano-digestion. The cell is then isolated in the optical trap (2) and monitored by multi-dimensional fluorescence imaging (3). Manipulation of the cell is carried out by one or more manipulators (not shown) and the post-digestion products are separated by a microfluidic separator (4) prior to detection (5) via 2D optical finger printing or multi-dimensional fluorescence imaging.

The microfluidic cell analyser is provided for the analysis of the outer surface of a cell, particularly for the analysis of the plasma membrane of a cell.

The analyser can be used to analyse one or more proteins, glycoproteins or lipids in or associated with the plasma membrane of the cell.

In use, a single cell is introduced into the optical cell trap. For the purposes of this invention, the optical trap comprises a laser and a focusing lens. Manipulation of the cell within the optical trap is achieved by a controller which contains the focused laser beam's position, polarization, phase and profile and which is used to realize the functions given above. This controller can be included on the low magnification side of the lens where the laser beam is collimated.

The position of the cell within the optical trap is monitored by an imager, such as a multi-dimensional fluorescence imager, which provides information regarding the status of the cell, this information being fed back to the controller.

The term “optical trapping” refers to the process whereby cells of a high dielectric constant are naturally attracted to regions of high electric field, for example the maximum electric field produced in the focus of a laser beam. Alternatively optical trapping is effected by forming a dark spot in the centre of the focused beam to trap cells (with appropriate dielectric properties) which are repelled by the field, wherein the object is forced into the dark centre. The exact form of the beam focus e.g. to form lines or curves, can be used to trap extended or non-spherical cells.

Alternatively the cell may be trapped by other methods, such as electrostatics.

Manipulation of the cell within an optical trap can be achieved by for example using the focused laser as a controller to move the spot in three dimensions to change the position of the cell. Alternatively adjusting the separation of multiple spots can stretch or compress a cell and rotating the polarization or pattern of spots can rotate the cell. Similarly beam profiles with so-called “angular momentum” can be used to rotate the cell.

The cell is trapped to ensure that the cell remains stationary within the solvent flow, during analysis. Because of the dynamic fluid environment as well as the changing size and characteristics of the cell as it is manipulated, adaptive control is required to maintain the cell and its components in position. This is achieved for the present invention using programmable diffractive optics to dynamically control a near infra-red laser trapping beam in real time. Programmable diffractive optics is achieved with a Spatial Light Modulator (SLM), which provides a means of altering the phase, amplitude or polarization of light reflected off or passing through it under external electrical or optical control. The device is programmed with an appropriate computer generated hologram and with the aid of appropriate external optical components. The SLM is configured to impart an arbitrary phase, polarization and amplitude distribution onto a laser beam. In this way the device allows the dynamic control of the focused profile of the laser beam in the optical trap.

Other methods of controlling the laser beam for the purpose of this invention include scanning mirrors or acousto-optic deflectors. These are capable of temporally multiplexing (scanning) the beam between multiple positions.

The programmable diffractive optics allows the use of complex beam patterns to effectively trap the whole of the cell and its contents as the manipulation process proceeds. The set-up is inherently adaptive to be able to compensate for changes in the optical properties of the flowing cell medium and the programmable nature permits the cell to be moved or rotated. This feature interacts with active control of the flow dynamics to permit selective targeting of specific features on the cell membrane. Interactive use of the system with an operator allows the dynamic manipulation of the cell, so that the selective features are positioned by rotating or translating the trapped cell appropriately into the fluid flow.

The trapping and manipulation of the single cell may be combined with an imager such as online multi-dimensional fluorescence imaging (MDFI: resolving fluorescence in 2 or 3 spatial dimensions and with respect to some or all of lifetime, wavelength and polarisatrion) to provide an interactive system for manipulating and monitoring the cell. This provides interactive feedback to enhance the selective targeting of specific features in the cell membrane. The online multi-dimensional fluorescence imaging can use either endogenous autofluorescence or appropriate fluorescence labels to provide an interactive system for manipulating and monitoring the cell. Alternatively or in addition, the online multi-dimensional fluorescence imaging can provide an interactive system that may also be coupled to the selective dissolution of the plasma membrane. Furthermore, the online multi-parameter fluorescence imaging can be used to monitor the process and assist with the readout of the analytes sperted by the microfluidics system.

Cells can be introduced into the microfluidic cell analyser through an input reservoir. They can be moved through the analyser using suitable forces, such as hydrodynamic forces and/or electrokinetic forces, through a microchannel network in the analyser to a suitable position where they can be optically trapped. The microchannels have dimensions (channel width and channel height) so as to allow facile passage of the cell through the channel without interaction with microchannel surfaces and walls.

The single cell can be introduced into the trap using an aspiration assembly in conjunction with micromanipulators. This will allow the cell to be transferred from its growth medium to the microfluidics device where lamellar streams will then be used to deliver the cell to the optical trap.

The microfluidic cell analyser can additionally comprise a microfluidic separator located between the single cell trap and the detection zone. Separation of the components subsequent to manipulation can be carried out by size, isoelectric focussing, mass and/or mass/charge. Alternatively the cell can be analysed in the optical trap.

The outer surface of the trapped cell undergoes manipulation by a manipulator. In particular, the plasma membrane is manipulated by the manipulator. Manipulation can be carried out by physical or chemical means. In particular, the manipulator can cause the outer surface of the cell to be exposed to an enzyme (such as a lipase), a lipid, a detergent, sonication and/or physical agitation (e.g. laser dissection). The chemical agents can be delivered to defined locations (subcellular microdomains) on the membrane surface using multiple laminar streams within the microchannel. By controlling both the position and angular orientation of the cell relative to the reagent stream it is possible to either digest the plasma membrane in its entirety or selectively target subcellular microdomains thereby providing spatial information regarding the distribution of chemical moieties within the plasma membrane. Material released from the membrane is collected in continuous flow and directed downstream for separation and analysis. Alternatively, the biology of the cell may be manipulated to promote areas of stress within the plasma membrane (i.e. via the initiation of exocytosis) by the delivery of appropriate chemical agents. The cell may further be incubated with one or more hormones, proteins, etc.

In particular a small plug (10 pL-10 nL) of material that can digest the membrane can be introduced into the analyser, for example using electrokinetic control and/or ‘tee-injectors’ upstream of the optical trap. When the plug contacts the cell, the cell is rotated to allow the digested material to be released and motivated downstream towards the microfluidic separator.

The plasma membrane may be manipulated over the whole of the external cell surface. Alternatively, a portion of the plasma membrane may be manipulated. In particular, manipulation may be directed to the lipids and/or proteins comprising the plasma membrane.

The analytical device of the first aspect of the invention is directed to the analysis of the outer cell surface, more specifically to the plasma membrane. The manipulation of the plasma membrane does not result in the lysis of the cell. It will be appreciated that disruption of localized areas of the plasma membrane may result in the formation of a weakened area or a disruption of the membrane through which some of the cell contents may pass. However, such increase in the permeability of the membrane does not directly lead to full lysis of the cells.

Once manipulation of the cell has been completed, the cell can subsequently be lysed and the internal contents of the cells separated from the plasma membrane contents. The separated plasma membrane can then be further digested as required. Alternatively, the cell can be retained in an intact form and the cell separated from any digestion products.

The analyser comprises a detector for detecting the outer surface of the single cell, or components of the outer surface of the single cell. The cell is preferably detected by multiparameter fluorescence imaging and/or optical finger printing.

In particular, the plasma membrane and/or components thereof can be analysed using multiparameter fluorescence imaging (MDFI). MDFI is realized using a high-speed quasi-wide-field multiphoton microscope to provide rapid optical sectioning together with the ability to rapidly acquire excitation and emission spectral profiles as well as fluorescence lifetime data. The MDFI offers at least three important capabilities. First, using appropriate fluorescence labels (either genetically expressed or tagged with antibodies), it enables the direct observation of specific components in the cell membrane and their tracking through the microfluidic separation system. Secondly, the use of multi-photon excitation combined with spectrally resolved FLIM provides unprecedented contrast of autofluorescence, permitting the entire process to be controlled and monitored without using exogenous fluorescence labels that could compromise some samples. Finally, the MDFI provides one way to achieve the optical read-out of the microfluidic separation, distinguishing different proteins etc.

Alternatively, or in addition optical readout can be obtained with single molecule sensitivity using multi-dimensional fluorescence imaging and/or optical fingerprinting technologies. While MDFI essentially probes the spectroscopy associated with the electronic energy level structure, the optical fingerprinting technology is a vibrational spectroscopic tool that can directly resolve the individual bonding patterns in molecules.

Optical fingerprinting is achieved using an optical analog of 2D NMR. This optical analog uses two infra-red laser beams to excite two vibrations, and the vibrational coupling can then be monitored in a variety of ways. In a preferred feature of the invention, detection is obtained using DOubly Vibrationally Enhanced (DOVE) spectroscopy. DOVE reads out the vibrational coupling using a third laser pulse. The invention also encompasses extensions of DOVE such as TRIVE which use three infra-red pulses to excite three vibrations, and a fourth pulse to read out the coupling. This measurement of coupling is analogous to NOESY or COESY, NMR methods that measure spin-spin coupling. The vibrational coupling spectrum is projected over a multi-dimensional spectral space, one for each IR laser beam, and also one time dimension for the timing between each IR pulse. In this way the overdense and congested IR spectrum is thinned by looking at couplings only and being projected over a higher dimensional space, as is the case in 2D NMR.

The microfluidic analyser may comprise two or more single cell traps and/or two or more detection zones. This allows a high through put approach to single cell analysis. In particular, one detector (for example a multiparameter fluorescence arrangement) can be used to track two or more single cell traps and/or two or more detection zones. The outputs of the multiple detection zones can be combined to allow high throughput read out via optical fingerprinting. This approach allows the analysis of large quantities of one or more plasma membrane components very rapidly.

Single cell methods can be scaled out by providing two or more microfluidic cell analysers. The first aspect of the invention therefore encompasses an array of microfluidic cell analysers comprising two or more microfluidic cell analysers. The analysers are provided in parallel or in series.

It will be understood by the skilled person that any appropriate component can be used for the purposes of the present invention. In particular, the single cell trap, the manipulator, the detection zone or the detector can be any appropriate component.

The single cell for the purpose of the first aspect is obtained from a largely hetereogeneous or homogeneous population of cells. Such cells include mammalian or non-mammalian cells, including plant or animal cells. The cells can be isolated from a plant or animal or produced in vitro. The cells may be native cells, or genetically, chemically or biologically manipulated. Such cells include dendrites, mucosal cells, and epithelial cells.

The second aspect of the invention provides a method of single cell analysis comprising trapping a single cell, manipulating the outer surface of the cell and analysing the manipulated cell surface. The outer cell surface is preferably the plasma membrane.

The outer surface of the cell can be manipulated by exposure to one or more hormones, proteins, enzymes, lipids, detergents, sonication, and/or physical agitation. The online multi-dimensional fluorescence imaging can allow the degree and position of manipulation of the outer surface of the cell to be determined. The manipulation can then be analysed by 2D optical finger printing or multi-dimensional fluorescence imaging.

In particular, the second aspect relates to a method of single cell analysis comprising pre-separation of the cell sample by controlled microfluidic nano-digestion, trapping of the cell in an optical trap, monitoring of the cell by multi-dimensional fluorescence imaging, manipulation of the cell by a manipulator, separation of the resulting components of the cell by microfluidic separation and detection via 2D optical finger printing or multi-dimensional fluorescence imaging.

All preferred features of the aspects of the invention apply to all other aspects mutatis mutandis.

1. A microfluidic cell analyser comprising a single cell trap, a manipulator arranged to manipulate the outer surface of a cell in the trap, a detection zone in communication with the single cell trap and a detector.

2. A microfluidic cell analyser as claimed in claim 1 further comprising a microfluidic separator between the single cell trap and the detection zone.

3. A microfluidic cell analyser as claimed in claim 1 wherein the trap is an optical trap.

4. A microfluidic cell analyser as claimed in claim 3 wherein the analyser comprises a controller which is capable of controlling the position of a cell within the optical trap, and an imager capable of providing information regarding the status of the cell and feeding the information back to the controller.

5. A microfluidic cell analyser as claimed in claim 1 wherein the detector provides multiparameter fluorescence imaging and/or optical finger printing.

6. A microfluidic cell analyser as claimed in claim 1 wherein the detector is a high-speed quasi-wide-field multiphoton microscope.

7. A microfluidic cell analyser as claimed in claim 1 wherein the detector provides analysis by DOubly Vibrationally Enhanced (DOVE) spectroscopy.

8. A microfluidic cell analyser as claimed in claim 1 comprising two or more single cell traps.

9. A microfluidic cell analyser as claimed in claim 1 comprising two or more detectors.

10. A microfluidic cell analyser as claimed in claim 1 wherein the trap is monitored by an online multi-dimensional fluorescence imager.

11. A method of single cell analysis comprising trapping a single cell, manipulating the outer surface of the cell and analysing the manipulated cell surface or components released from the outer surface of the cell.

12. A method as claimed in claim 11 wherein the trapped cell is monitored by online multi-dimensional fluorescence imaging.

13. A method as claimed in claim 12 wherein the online multi-dimensional fluorescence imaging uses endogenous autofluorescence or fluorescence labels.

14. A method as claimed in claim 11 wherein monitoring of the trapped cell provides a feedback system for manipulating and monitoring the cell, wherein in the feedback system information regarding the status of the cell provided by online multi-dimensional fluorescence imaging is fed back to a controller which is used to control the position and orientation of the trapped cell.

15. A method as claimed in claim 14 wherein the feedback system is coupled to the selective dissolution of the plasma membrane of the cell.

16. A method as claimed in claim 11 wherein the outer surface of the cell is manipulated by exposure to one or more selected from the group comprising hormones, proteins, enzymes, lipids, detergents, sonication, and physical agitation.

17. A method as claimed in claim 11 wherein the cell is obtained from a hetereogeneous or homogeneous population of cells.

18. A method as claimed in claim 11 wherein the cell is selected from the group comprising a mammalian and non-mammalian cell.

19. A method as claimed in claim 11, wherein the cell is trapped in an optical trap.

20. A method as claimed in claim 11 wherein the manipulated cell surface is analysed by 2D optical finger printing or multi-dimensional fluorescence imaging.

21. A method as claimed in claim 11 wherein a cell sample is pre-separated by controlled microfluidic nano-digestion, prior to trapping of the cell.

22. A method as claimed in claim 11 wherein the components of the cell are separated by microfluidic separation prior to analysis.

23. A method as claimed in claim 11 comprising pre-separation of the cell sample by controlled microfluidic nano-digestion, trapping of the cell in an optical trap, monitoring of the cell by multi-dimensional fluorescence imaging, manipulation of the cell by a manipulator, separation of the resulting components of the cell by microfluidic separation and detection via 2D optical finger printing or multi-dimensional fluorescence imaging.

24. (canceled)

25. (canceled)
